(19) United States
(12) Patent Application Publication
(10) Pub. No.: US 2018/0010090 A1
(43) Pub. Date: Jan. 11, 2018

(54) FORMULATIONS AND METHODS FOR INCREASED RECOMBINANT PROTEIN PRODUCTION

(71) Applicant: Bayer HealthCare LLC, Research Triangle Park, NC (US)

(72) Inventors: Yuval SHIMONI, Berkeley, CA (US); Volker MÖHRLE, Koln (DE); Venkatesh SRINIVASAN, Campbell, CA (US); Ricaredo MATANGUIHAN, Walnut Creek, CA (US)

(21) Appl. No.: 15/699,088

(22) Filed: Sep. 8, 2017

Related U.S. Application Data
(62) Division of application No. 14/770,064, filed on Aug. 24, 2015, now abandoned.
(60) Provisional application No. 61/769,402, filed on Feb. 26, 2013.

(51) Int. Cl. C12N 5/00; C07K 14/755; C12P 21/02

(52) U.S. Cl. CPC C12N 5/0018; C12P 21/02; C07K 14/755; C12N 2500/14; C12N 2500/34

(57) ABSTRACT

Formulations and methods to increase the production of recombinant proteins, and other aspects, are disclosed. The formulations and methods relate to increasing mannose or calcium concentration, or both, in a cell culture medium formulation for culturing cells that express recombinant proteins. In some embodiments, a mammalian cell culture medium formulation is provided that has at least one of mannose at about 3.5 g/L or more and calcium in a range from about 1.5 mM to about 9.5 mM. Numerous other aspects and/or embodiments are provided.

### RELATED APPLICATIONS

The present application claims priority from U.S. Provisional Patent Application Ser. No. 61/769,402, filed Feb. 26, 2013, entitled “FORMULATIONS AND METHODS FOR INCREASED RECOMBINANT PROTEIN PRODUCTION” (Attorney Docket No. BHC125022US (BH-023/L)), which is hereby incorporated herein by reference in its entirety for all purposes.

### BACKGROUND

Cell culture systems can be used to produce recombinant proteins in cell culture medium formulations that include nutrients to promote cell growth. Example cell culture medium formulations include DMEM/F12, RPMI (e.g., RPMI 1640), MEM, DMEM, F-12, mouse ES cell basal medium, L-15, IMDM, McCoy's 5A medium, and VeroPlus SFM.

In production of recombinant proteins, including in commercial production, it is desirable to increase the level of recombinant protein production while preserving product quality.

Accordingly, there is a need for cell culture medium formulations and methods that increase recombinant protein production without decreasing the quality of the product.

### SUMMARY

In some embodiments, a mammalian cell culture medium formulation is provided. The formulation has at least one of mannose at about 3.5 g/L or more and calcium in a range from about 1.5 mM to about 9.5 mM.

In one or more embodiments, a method of producing a recombinant protein in cell culture is provided. The method includes culturing recombinant protein expressing cells in a cell culture medium having at least one of mannose at about 3.5 g/L or more and a stabilizer of the recombinant protein, such as calcium in a range from about 1.5 mM to about 9.5 mM. In certain embodiments, the method results in an increase in the production of the recombinant protein. In certain embodiments, the method results in an increase in the production of the recombinant proteins without compromising the quality of the recombinant proteins produced.

Numerous other aspects are provided in accordance with these and other embodiments. These and other features of the present teachings are set forth herein.

### DRAWINGS

The skilled artisan will understand that the drawings, described below, are for illustration purposes only. The drawings are not intended to limit the scope of the present teachings in any way.

FIG. 1 is a flowchart illustrating a method of increasing recombinant protein production in cell culture systems in accordance with various embodiments.

FIG. 2 shows graphically that an increase in mannose concentration from 3 grams/liter (“g/L”) to 5 g/L increased recombinant human factor VIII (“rhFVIII”) titer by 25% in a 1 L perfusion bioreactor cell culture in accordance with various embodiments.

FIG. 3 shows graphically that an increase of mannose concentration from 3 g/L to 5 g/L resulted in ˜37% increase in rhFVIII titer in a 15 L perfusion bioreactor cell culture in accordance with various embodiments.

FIG. 4 shows graphically the results demonstrating highest impact on rhFVIII titer (˜19% increase) at the tested condition of 5 millimolar (“mM”) calcium chloride in roller tube (repeat-batch) experiments in accordance with various embodiments.

FIG. 5 shows graphically that an increase in calcium concentration from 1 mM to 5 mM increased rhFVIII titer by ˜27% in a 1 L perfusion bioreactor cell culture in accordance with various embodiments.

FIG. 6 shows graphically that increasing calcium concentration from 1 mM to 5 mM increased rhFVIII titer by ˜29% in a 15 L perfusion bioreactor cell culture in accordance with various embodiments.

FIGS. 7A-B show graphically the results of shifting from control medium (containing 1 mM calcium chloride and 3 g/L mannose) to medium enriched for both components—containing 5 mM Calcium chloride and 5 g/L mannose—increased rhFVIII titer by 29% and the effect is reversible in accordance with various embodiments.

FIGS. 8A-B show the design of a 15 L perfusion bioreactor campaign (A) and the resulting potency data (B), in accordance with various embodiments.

FIG. 9 shows graphically the results of manipulating the sugar content of a cell culture formulation on production levels of rhFVIII, showing the average values of potency using mannose-containing and mannose free medium, in accordance with various embodiments.

### DESCRIPTION OF VARIOUS EMBODIMENTS

As stated, increasing production level of protein expressing cells cultured in a cell culture medium formulation without adversely affecting protein product quality is a challenge. In accordance with one or more embodiments described herein, by providing additional nutrients such as mannose and/or a stabilizer such as calcium to a cell culture medium, an improved cell culture medium formulation can be created. In some embodiments, the improved cell culture medium formulation can increase production level of protein expressing cells cultured using the cell culture medium formulation with little or no detectable impact to product quality. Methods of forming and/or using such cell culture medium formulations are also provided.

### Example Cell Culture Medium Formulations

As stated above, in various embodiments, increase in the production of recombinant proteins in cell culture medium formulations can be achieved by increasing the concentration of mannose and/or the concentration of a stabilizer of a recombinant protein, such as calcium, or both in the formulations.

In one aspect, a cell culture medium formulation (e.g., a cell culture medium composition) is provided that includes at least one of mannose at about 3.5 g/L or more (or, in certain embodiments, at about 4 g/L, about 5 g/L, about 6 g/L, or about 7 g/L or more) and calcium in a range from about 1.5 mM to about 9.5 mM or more (or, in certain embodiments, at about 2 mM, about 3 mM, about 4 mM, about 5 mM, about 6 mM, about 7 mM, about 8 mM, about 9 mM, or about 9.5 mM or more). Other cell culture medium formations can be employed.

The cell culture medium formulation, prior to the addition of at least one of mannose at about 3.5 g/L or more and calcium in a range from about 1.5 mM to about 9.5 mM, can be any cell culture medium formulation. For example, in certain embodiments, the cell culture medium formulation can include Dulbecco's Modified Eagle's Medium and Ham's F-12 Nutrient Mixture (DMEM/F12) in a suitable ratio such as 1:1, and at least one of mannose at about 5 g/L and calcium at about 5 mM.

Other cell culture medium formations can be employed in place of DMEM/F12 such as RPMI (e.g., RPMI 1640), MEM, DMEM, F-12, mouse ES cell basal medium, L-15, IMDM, McCoy's 5A medium, and VeroPlus SFM. In certain embodiments, the cell culture medium formulation is for culturing mammalian cells.

Example cell culture medium formulations provided herein include without limitation:
(a) mannose at about 3.5 g/L or more and calcium at less than about 1.5 mM or more than about 9.5 mM;
(b) mannose at less than about 3.5 g/L and calcium in a range from about 1.5 mM to about 9.5 mM;
(c) at least one of mannose in a range from about 4 g/L to about 5 g/L and calcium in a range from about 1.5 mM to about 9.5 mM;
(d) at least one of mannose at about 5 g/L and calcium in a range from about 1.5 mM to about 9.5 mM;
(e) mannose in a range from about 4 g/L to about 5 g/L and calcium at less than about 1.5 mM or more than about 9.5 mM;
(f) mannose at about 5 g/L and calcium at less than about 1.5 mM or more than about 9.5 mM;
(g) at least one of mannose at about 3.5 g/L or more and calcium in a range from about 2 mM to about 5 mM;
(h) at least one of mannose at about 3.5 g/L or more and calcium at about 5 mM;
(i) mannose at less than about 3.5 g/L and calcium in a range from about 2 mM to about 5 mM;
(j) mannose at less than about 3.5 g/L and calcium at about 5 mM; and/or
(k) DMEM/F12 in 1:1 ratio, and at least one of mannose at about 5 g/L and calcium at about 5 mM.
   Other formulations can be employed.

### Methods of Forming/Using Cell Culture Medium Formulations

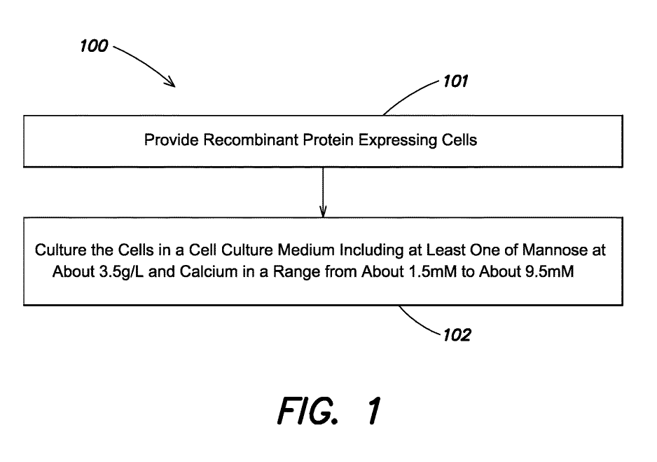

Methods to produce a recombinant protein in cell culture will now be described with reference to FIGS. 1-8 below.

FIG. 1 illustrates a flowchart of a method 100 for producing a recombinant protein in cell culture in accordance with certain embodiments provided herein. With reference to FIG. 1, method 100 begins with Block 101 in which recombinant protein expressing cells are provided.

Example recombinant protein expressing cells can include, for example, any eukaryotic or prokaryotic cells, including mammalian cells, plant cells, insect cells, yeast cells, bacterial cells or the like. In certain embodiments, the cells are mammalian cells. Example mammalian cells include baby hamster kidney (BHK) cells, Chinese hamster ovary (CHO) cells, hybrid of kidney and B cells (HBK) cells, human embryonic kidney (HEK) cells, and NS0 cells.

The recombinant protein expressing cells can be any cells making any biologic protein products. For example, the cells can be recombinant cells that are engineered to express one or more recombinant protein products; and/or recombinant cells that express antibody molecules.

The product of the recombinant protein expressing cells can be any protein product, including recombinant protein products such as coagulation factors (a protein in the blood coagulation pathway), including for example factor VII, factor VIII, factor IX and factor X. For example, the recombinant protein expressing cells can be mammalian cells expressing factor VIII.

The factor VIII could be variants of factor VIII, such as genetic variants, which could be created by making genetic variation of the rFVIII gene constructs, resulting in, for example, B-domain deleted factor VIII and mutated factor VIII. The factor VIII variants include, for example, variants of factor VIII modified post expression, such as, for example, pegylated FVIII and FVIII with covalently attached polyethylene glycol (PEG). Factor VIII variant can also include fusion proteins with co-expressed binding elements.

In certain embodiments, the recombinant protein product of the recombinant protein expressing cells can be a glycoprotein. In some embodiments, the recombinant protein is secreted. Any suitable source of and/or method for forming recombinant cells expressing recombinant proteins can be employed.

In Block 102, the recombinant protein expressing cells are cultured in a cell culture medium formulation (i.e., composition) that includes at least one of mannose at about 3.5 g/L or more and calcium in a range from about 1.5 mM to about 9.5 mM. As used herein, a cell culture medium can include a tissue or cell culture fluid, tissue or cell culture medium or media, or the like.

The cell culture medium formulation, prior to the addition of at least one of mannose at about 3.5 g/L or more and calcium in a range from about 1.5 mM to about 9.5 mM, can be any cell culture medium formulation. For example, in certain embodiments, the cell culture medium formulation can include Dulbecco's Modified Eagle's Medium and Ham's F-12 Nutrient Mixture (DMEM/F12) in a suitable ratio such as 1:1, and at least one of mannose at about 5 g/L and calcium at about 5 mM.

Other cell culture medium formations can be employed in place of DMEM/F12 such as RPMI (e.g., RPMI 1640), MEM, DMEM, F-12, mouse ES cell basal medium, L-15, IMDM, McCoy's 5A medium, and VeroPlus SFM. In certain embodiments, the cell culture medium formulation is for culturing mammalian cells.

In various embodiments, the cell culture medium formulation can be a media composition based on a commercially available DMEM/F12 formulation manufactured by Sigma-Aldrich Fine Chemicals (SAFC, Lenexa, Kans.) or Life Technologies (Grand Island, N.Y.) supplied with other supplements such as iron, Pluronic F-68, or insulin, and can be essentially free of other proteins. Other base media compositions may be employed.

Complexing agents histidine (his) and/or iminodiacetic acid (IDA) can be used, and/or organic buffers such as MOPS (3-[N-Morpholino]propanesulfonic acid), TES (N-tris[Hydroxymethyl]methyl-2-aminoethanesulfonic acid), BES (N,N-bis[2-Hydroxyethyl]-2-aminoethanesulfonic acid) and/or TRIZMA (tris[Hydroxymethyl]aminoethane) can be used; all of which can be obtained from SAFC (St. Louis, Mo.), for example. In some cases, the tissue culture media can be supplemented with known concentrations of these complexing agents and/or organic buffers individually or in combination. In some embodiments, a tissue culture fluid can contain EDTA, e.g., 50 μM, or another suitable metal (e.g., iron) chelating agent. Other compositions, formulations, supplements, complexing agents and/or buffers can be used.

The cell culture medium formulation can include amino acids, which can include, for example, any of the naturally occurring amino acids.

The cell culture medium formulation can include salts, which can include potassium chloride, magnesium sulfate, sodium chloride, sodium phosphate, magnesium chloride, cupric sulfate, ferrous sulfate, zinc sulfate, ferric nitrate, selenium dioxide, calcium chloride and/or other salts suitable for use in a cell culture medium formulation.

The cell culture medium formulation can include vitamins, which can include biotin, choline chloride, calcium pantothenate, folic acid, hypoxanthine, inositol, niacinamide, vitamin C, pyridoxine, riboflavin, thiamine, thymidine, vitamin B-12, pyridoxal, putrescine and/or other vitamins suitable for use in a cell culture medium formulation.

The cell culture medium formulation can include one or more components other than those listed above (“other components”), which can include dextrose, mannose, sodium pyruvate, phenol red, glutathione, linoleic acid, lipoic acid, ehanolamine, mercaptoethanol, ortho phophorylethanolamine and/or other components suitable for use in a cell culture medium formulation.

A common mammalian cell culture medium formulation is DMEM/F12. DMEM/F12 is a 1:1 mixture of Dulbecco's Modified Eagle's Medium (DMEM) and Ham's F-12 Nutrient Mixture. DMEM/F12 medium is available from many commercial sources and is often used in the production of recombinant proteins such as rhFVIII. The complete component composition of DMEM/F12 is freely available (e.g., ATCC Cat #30-2006) (Table 1). DMEM/F12 (1:1) typically contains 1.05 mM (0.11665 g/L) of freely soluble CaCl₂ (anhydrous). D-mannose is not a component of the DMEM/F12 (1:1) formula; D-glucose is present (as a carbohydrate source) at about 3 g/L.

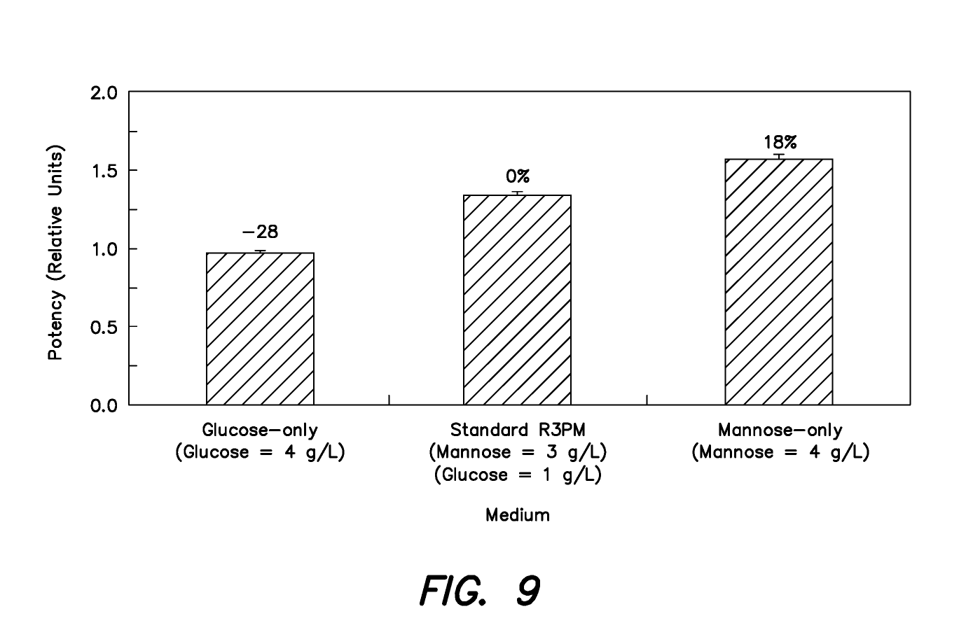

In certain embodiments, a formulation is provided comprising DMEM/F12 and mannose at about 3 g/L or less. For example, a formulation with DMEM/F12 (with glucose at 1 g/L) and mannose at 3 g/L (with 4 g/L of total sugar) can result in an increase in rhFIII titer in a cell culture by about 28% as compared to a cell culture with DMEM/F12 without any mannose, but with 4 g/L of glucose (4 g/L total sugar). In certain embodiments, a formulation with DMEM/F12 with 4 g/L mannose (4 g/L total sugar) but no glucose, can result in an increase in rhFVIII titer in a cell culture by about 18% compared to a cell culture with DMEM/F12 with 3 g/L mannose and 1 g/L glucose (4 g/L total sugar) See, for example, FIG. 9 which illustrates graphically the results of manipulating the sugar content of a cell culture formulation on production levels of rhFVIII.

Mannose is a sugar monomer and an epimer of glucose. Mannose is involved in cell metabolism. It is incorporated into a protein post-translationally during glycoprotein biosynthesis. Oligosaccharides attached to glycoproteins can assist in the proper folding of the nascent protein and help protect the mature proteins from proteolysis (Hebert and Molinari, Physiol. Rev. 87: 1377-1408 (2007)). Typical N-linked oligosaccharides contain mannose, as well as N-acetylglucosamine and usually have several branches, sometimes with terminal negatively charged sialic acid residues. This structural modification is an important quality attribute for many glycoproteins, including FVIII, which can impact the molecule's biogenesis, secretion and stability and pharmacokinetic/dynamic (PK/PD) properties.

While eukaryotic cells are capable of converting glucose into mannose in a process where fructose-6-phosphate is converted to mannose-6-phosphate by Mannose-6 phosphate isomerase, in some cell types, most of the mannose for glycoprotein biosynthesis is derived from mannose, not glucose (Alton et al., Glycobiology 8 (3) 285-295 (1998)).

The stabilizer of a recombinant protein can be anything that stabilizes a recombinant protein from, for example, degradation. Examples of stabilizers include calcium and manganese.

Calcium ions play an important role stabilizing FVIII coagulation activity by stabilizing the quaternary structure of the FVIII complex (Switzer et al., The Journal of Clinical Investigation 60: 819-828 (1977); Mikaelsson et al. Blood 62(5): 1006-1015 (1983)). Calcium and manganese have been shown to promote FVIII activity by binding to both heavy and light chains thus modulating the conformation of the heterodimer (reviewed in Fay, Blood Rev. 18: 1-15 (2004)). It has been suggested that calcium (and/or manganese) is required to promote the active conformation of FVIII.

Any suitable cell culture system for culturing cells can be employed using an embodiment formulation and/or embodiment method. The cell culture system can be a mammalian cell culture system. The cell culture system can be a bioreactor cell culture system, including a perfusion bioreactor cell culture system. The cell culture system can include a small-scale culture system such as a tissue culture flask or roller bottle, and/or large-scale cell culture systems such as bioreactor cell culture systems. Example cell culture medium can be further supplemented by serum, including bovine serum, horse serum, calf serum, fetal calf serum, and/or fetal bovine serum. Example cell culture medium can be further supplemented by human serum and/or human plasma protein fraction.

A bioreactor cell culture system can include (1) recombinant protein expressing cells; and (2) a cell culture medium formulation selected from (a) a formulation comprising at least one of mannose at about 3.5 g/L or more and calcium in a range from about 1.5 mM to about 9.5 mM; (b) a formulation comprising mannose at about 3.5 g/L or more and calcium at less than about 1.5 mM or more than about 9.5 mM; (c) a formulation comprising mannose at less than about 3.5 g/L and calcium in a range from about 1.5 mM to about 9.5 mM; (d) a formulation comprising at least one of mannose in a range from about 4 g/L to about 5 g/L and calcium at about 1.5 mM to about 9.5 mM; (e) a formulation comprising at least one of mannose at about 5 g/L and calcium in a range from about 1.5 mM to about 9.5 mM; (f) a formulation comprising mannose in a range from about 4 g/L to about 5 g/L and calcium at less than about 1.5 mM or more than about 9.5 mM; (g) a formulation comprising mannose at about 5 g/L and calcium at less than about 1.5 mM or more than about 9.5 mM; (h) a formulation comprising at least one of mannose at about 3.5 g/L or more and calcium in a range from about 2 mM to about 5 mM; (i) a formulation comprising at least one of mannose at about 3.5 g/L or more and calcium at about 5 mM; (j) a formulation comprising mannose at less than about 3.5 g/L and calcium in a range from about 2 mM to about 5 mM; (k) a formulation comprising mannose at less than about 3.5 g/L and calcium at about 5 mM; and (1) a formulation comprising DMEM/F12 in 1:1 ratio, and including at least one of mannose at about 5 g/L and calcium at about 5 mM.

In some embodiments, through use of a cell culture medium formulation that includes at least one of mannose at about 5 g/L and calcium at about 5 mM, the production of the recombinant protein is increased. In certain embodiments, the production of the recombinant protein is increased without compromising the quality of the recombinant protein produced (e.g., when compared to the same or substantially the same cell culture medium without at least one of mannose at about 3.5 g/L or more and calcium in a range from about 1.5 mM to about 9.5 mM, or at any specific point(s) of these range(s) described herein). In certain embodiments, the increased production of the recombinant protein is sustained for up to about 130 days, or more.

Example cell culture systems and bioreactor cell culture systems for the production of recombinant proteins are described in the literature. Example perfusion culture systems for the production of recombinant Factor VIII are described in the literature at, for example, U.S. Pat. No. 6,338,964 entitled “Process and Medium For Mammalian Cell Culture Under Low Dissolved Carbon Dioxide Concentration,” and in Boedeker, B. G. D., Seminars in Thrombosis and Hemostasis, 27(4), pages 385-394.

The above-described formulations and methods can significantly increase plant capacity and reduce production costs. For example, in some embodiments, increase in cell culture productivity of up to ˜40% for rhFVIII has been observed (e.g., with productivity increase sustained for at least 3 months of continuous perfusion culture). Further, methods in accordance with certain embodiments are of relatively low complexity and cost to implement in a cGMP regulatory-agency compliant API production plant. For example, in various embodiments, there is no requirement for genetic manipulations or a change of cell line for an established recombinant protein product; no requirement for major changes to infrastructure or to production process; and/or no impact on product quality.

Aspects of the present teachings can be further understood in light of the following examples, which should not be construed as limiting the scope of the present teachings in any way.

### EXAMPLES

### Example 1: Increasing Mannose Resulted in Increased Production of Recombinant Proteins in a Cell Culture System

BHK-21 cells expressing rhFVIII were cultured in roller tubes (Shimoni et al., BioPharm International 23(8): 28-37 (2010)) with changes to the concentrations of existing DMEM/F12 media components. Increased rhFVIII titers (determined by assaying for potency) were observed when mannose levels were increased.

Experiments performed using a 1 L perfusion bioreactor system were followed by 15 L scale perfusion bioreactor studies. Results were generally consistent between the 1 L scale and the 15 L scale.

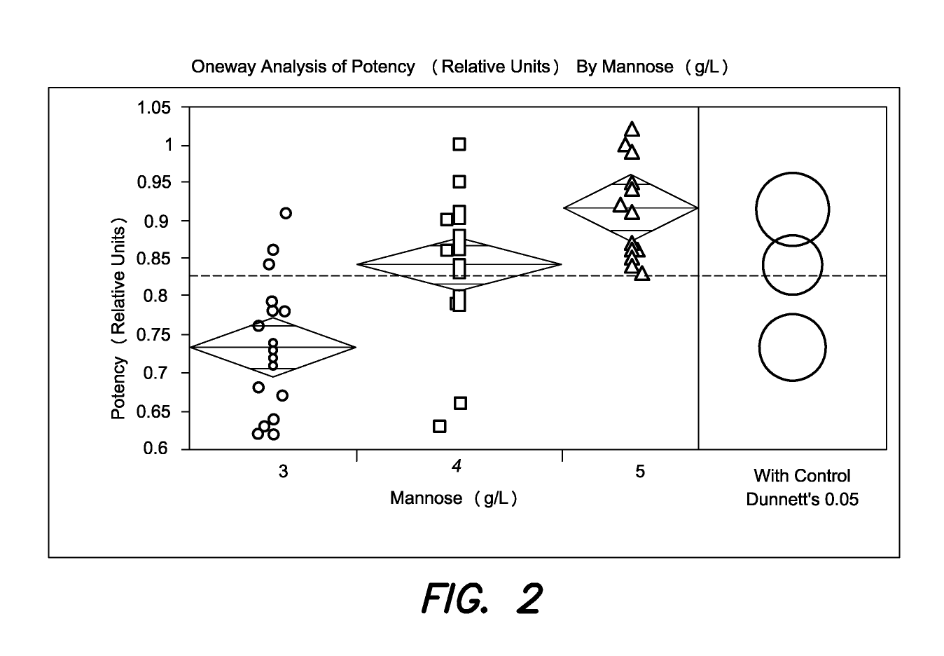

A range testing experiment performed at 1 L scale perfusion bioreactors demonstrated a dose dependent effect of mannose increase on titer, following inoculation and growth to steady state in standard medium containing 3 g/L mannose (control conditions). Cells were further continuously cultured for about 10 days each in the (standard) medium containing 3 g/L mannose, followed by 4 g/L and 5 g/L mannose (by switching the medium fed into the bioreactor). No other medium component was changed in this experiment. Samples were taken (processed and frozen) about daily for potency determination. Titer increased by ˜15% when mannose was increased from 3 to 4 g/L and by ˜25% (i.e., another ˜10%) when mannose was further increased to 5 g/L (FIG. 2).

Statistical analysis of the data from FIG. 2 demonstrated that the effect of mannose concentration increase on increasing potency/titer is significant (P-Value <0.0001).

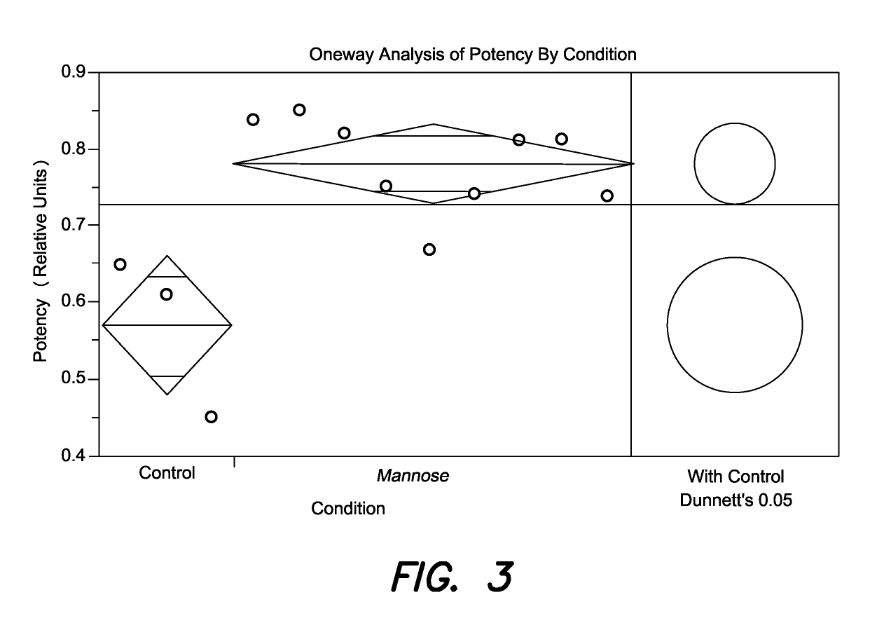

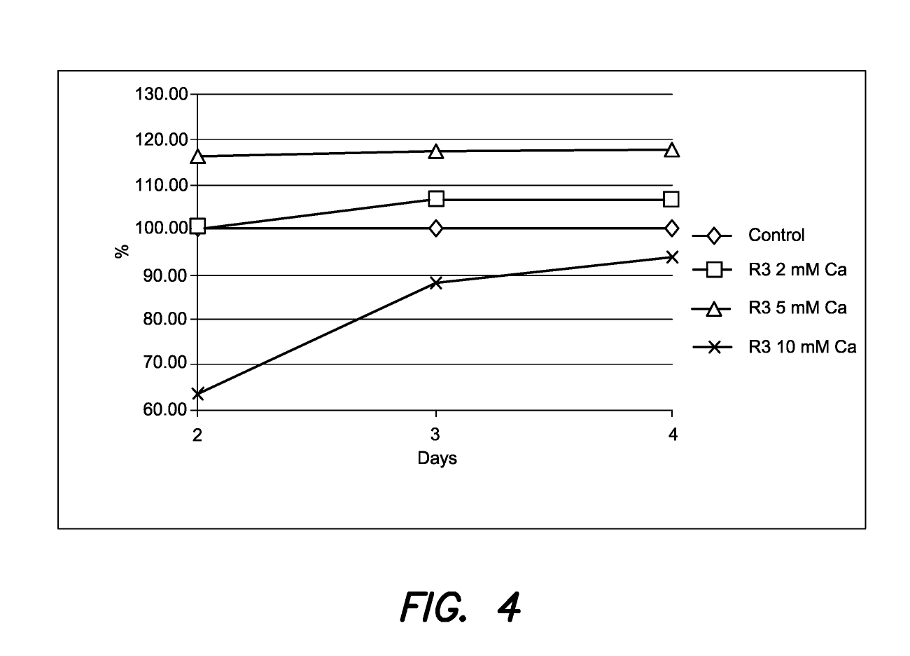

When the experiment was performed at 15 L scale, shifting the culture from media containing 3 g/L mannose to media containing 5 g/L mannose, with samples taken (processed and frozen) about daily, it resulted in ˜37% increase of titer (FIG. 3; 3 g/L mannose labeled as “Control” on the X-axis and 5 g/L mannose labeled as “Mannose” on the X-axis). In both experiments (performed at 1 L and at 15 L scale), statistical analysis shows that the effects of mannose increase are statistically significant. The effect of mannose on FVIII titer was observed within days of media switch and was sustained in the continuous (1 L and 15 L) perfusion culture systems.

Statistical analysis of the data from FIG. 3 demonstrates that the effect of mannose on increasing potency/titer is significant (P-Value <0.0012).

### Example 2: Increasing Calcium Resulted in an Increased Production of Recombinant Proteins in a Cell Culture System

BHK-21 cells expressing rhFVIII were cultured in roller tubes (Shimoni et al., BioPharm International 23(8): 28-37 (2010)). Increased rhFVIII titers (determined by assaying for potency) were observed when calcium levels were increased in the DMEM/F12 based medium.

A range finding experiment was performed in roller tubes to identify an optimal concentration of calcium for FVIII titer increase. Of the concentrations tested, 5 mM calcium chloride had the biggest impact (˜19% increase) on FVIII titer (FIG. 4: samples were collected on days 2, 3 and 4 over the 4-day experiment (X-axis) with titer (potency) given as % of control (1 mM calcium chloride) in the Y-axis). Calcium chloride concentrations ranging from 1 mM (control), 2 mM, 5 mM and 10 mM were tested. Calcium increase from 1 mM (control) to 2 mM increased FVIII titer by ˜8%, whereas at 5 mM titer increase by ˜19%. But at 10 mM, calcium had a negative impact on titer.

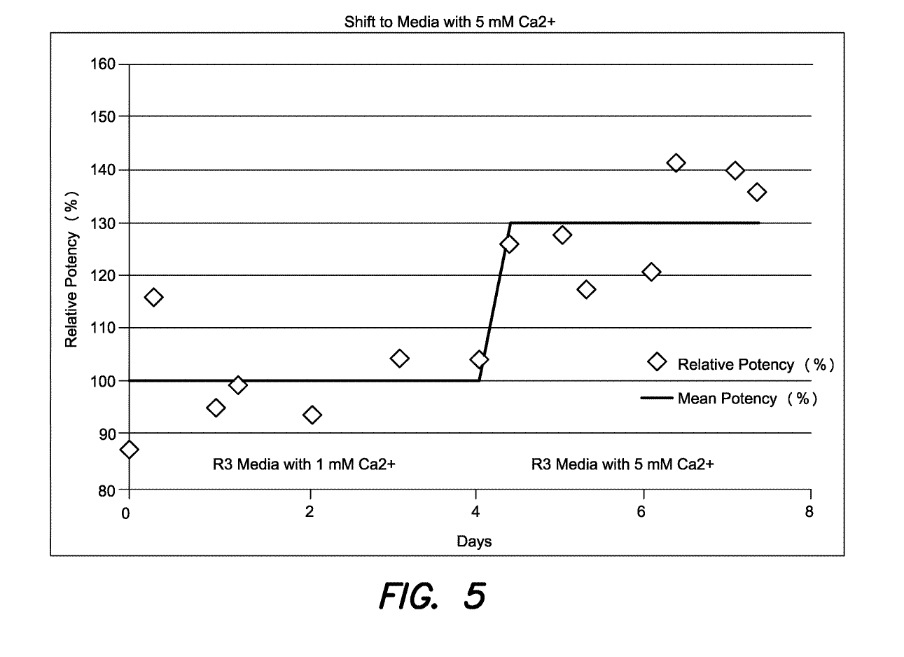

When cells grown in a 1 L perfusion bioreactor were shifted from 1 mM calcium chloride (control medium) to 5 mM calcium chloride containing medium, titer increased by ˜27% (FIG. 5). Cells were continuously cultured at steady state in a 1 L perfusion bioreactor for about 5 days in medium containing 1 mM calcium chloride and then shifted into and cultured for another ˜5 days in medium containing 5 mM calcium chloride. Samples were taken (processed and frozen) about daily for potency determination.

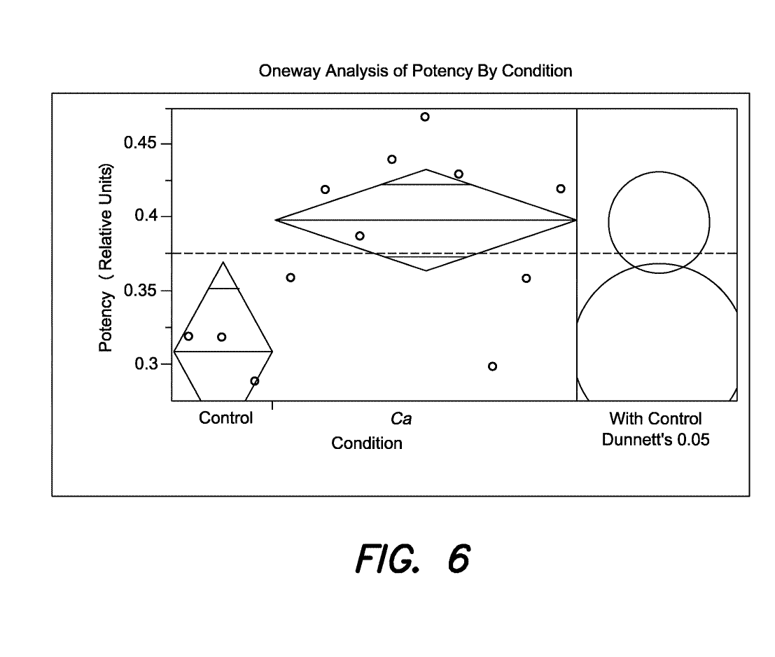

When a similar experiment was repeated at 15 L scale perfusion bioreactor, titer was ˜29% higher in media containing 5 mM calcium than in 1 mM calcium (FIG. 6: medium containing 5 mM calcium chloride labeled as “Ca” on the X-axis). Cells were continuously cultured at steady state in a 15 L perfusion bioreactor for about 3 days in medium containing 1 mM calcium chloride (“control”) and then shifted into and cultured for over a week in medium containing 5 mM calcium chloride. Samples were taken (processed and frozen) about daily for potency determination.

Both experiments, conducted at 1 L scale and at 15 L scale, were thus very consistent with each other, demonstrating a fast FVIII titer increase of 27-29%, once media was shifted from 1 mM to 5 mM calcium chloride. The higher titer was sustained throughout the duration of the experiment.

Material was harvested and concentrated by ultra-filtration at the end of the two 15 L runs described above: using medium containing 5 g/L mannose (FIG. 2) and using medium containing 5 mM calcium (FIG. 5).

Statistical analysis of the data from FIG. 6 demonstrates that the effect of calcium concentration increase on increasing potency/titer is significant (p-Value <0.0176).

### Example 3: Increasing Production of Recombinant Protein by Increasing Mannose or Calcium Concentration Did not Compromise Protein Quality

The frozen ultra-filtered culture harvest from Examples 1-2 (15 L bioreactor, approximately two-week long campaigns with each media type: A. 5 mM calcium; B. 5 g/L mannose) was then processed and FVIII was purified in several steps as previously described (Boedeker, Seminars in Thrombosis and Hemostasis 27(4): 385-394 (2001)) and finally assessed for various product quality attributes. rhFVIII material purified from both 5 g/L mannose containing medium and 5 mM calcium containing medium passed various product quality attributes including purity and integrity assessed by HPLC-SEC and SDS-PAGE/western blot based methods, potency, specific activity, various host-cell impurities (proteins and nucleic acids) and glycosylation patterns, indicating that the changes in mannose and calcium concentrations in the medium did not impact the FVIII product.

### Example 4: Increasing Mannose and Calcium Resulted in an Increased Production of Recombinant Proteins in a Cell Culture System

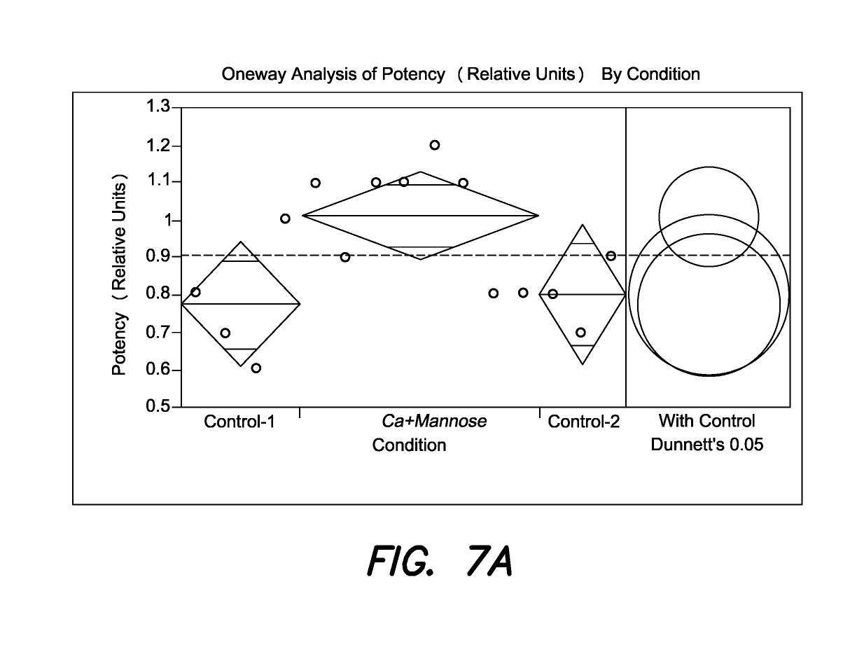

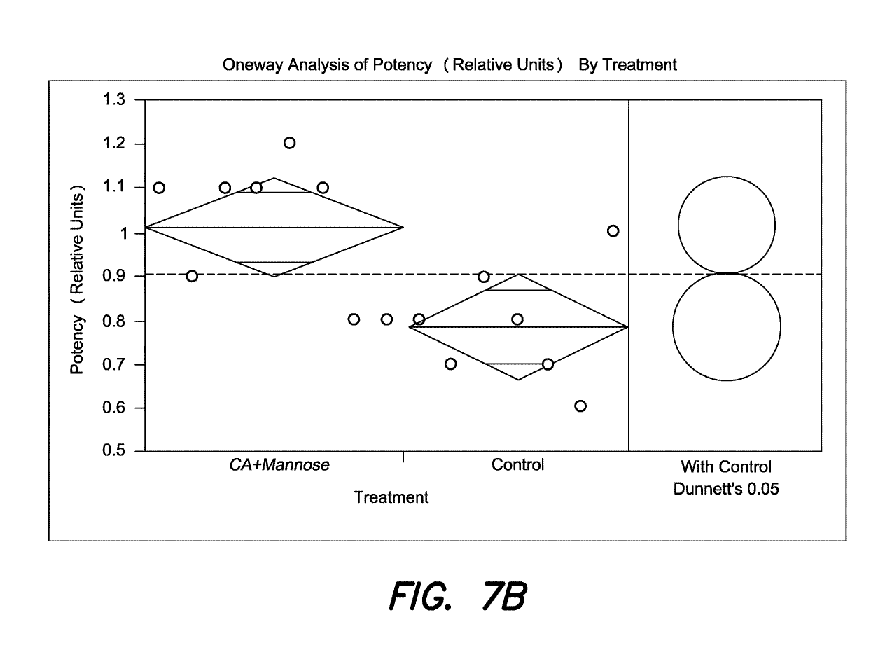

FIGS. 7A-7B show that a DMEM/F12 based media enriched for both (5 mM) calcium and (5 g/L) mannose had a higher beneficial effect on FVIII titer than each component alone. It also shows that the titer change occurred within a day and was reversible as the ˜29% increase in titer reversed to base line once the culture was returned to standard medium (containing 1 mM calcium chloride and 3 g/L mannose).

FIGS. 7A-B show graphically the results of shifting from control medium (containing 1 mM calcium chloride and 3 g/L mannose) to medium enriched for both components—containing 5 mM Calcium chloride and 5 g/L mannose—increased rhFVIII titer by 29%, and the effect is reversible. Cells continuously cultured in a 15 L perfusion bioreactor cell culture in standard medium (1 mM calcium and 3 g/L mannose; control-1) that were shifted to medium containing 5 mM calcium and 5 g/L mannose for about 9 days and then back to standard medium (control-2) demonstrated a fast (within one day) and reversible rhFVIII titer change. Conditions were “Control-1,” before shift and “Control-2” after shift to/from (respectively) 5 mM calcium chloride and 5 g/L mannose (“Ca+Mannose”) test medium. Samples were taken (processed and frozen) about daily for potency determination. Panel A, by “Condition.”

Statistical analysis of the data from FIGS. 7(A) and (B) demonstrates that the effect of concentration increase of mannose and calcium (together) on increasing potency/titer is significant. Statistical analysis of the data from FIG. 7(A) yields a p-Value <0.05. Statistical analysis of the data from FIG. 7(B) yields a p-Value <0.0102.

### Example 5: Increasing Production of Recombinant Protein by Increasing Mannose and Calcium Concentrations Did not Compromise Protein Quality

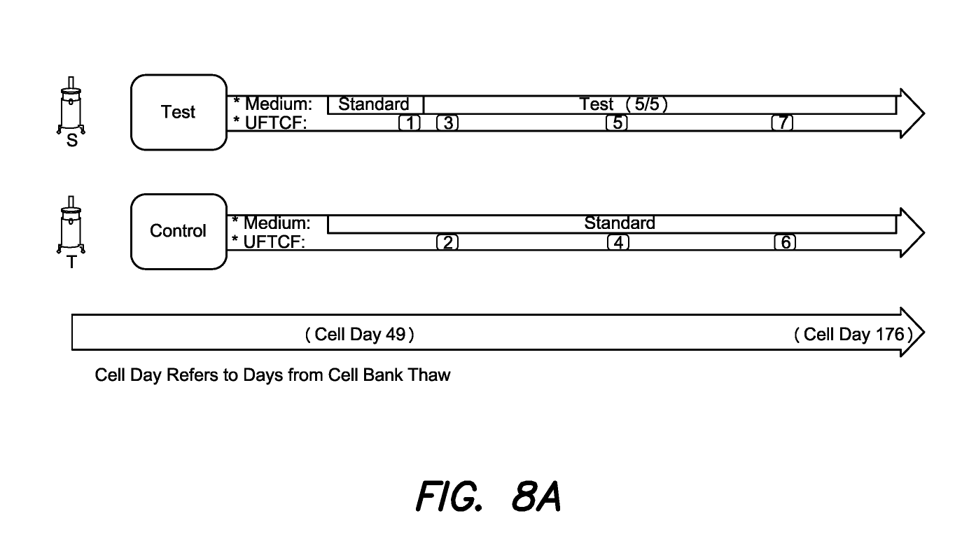

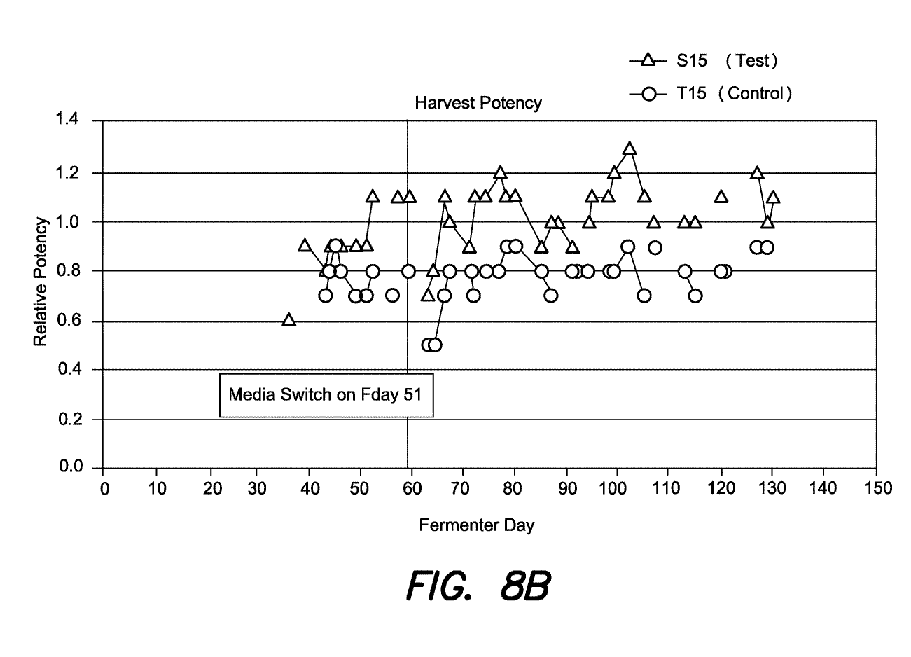

To verify that the effects are sustained over a campaign lasting over 130 days in perfusion culture, two bioreactors were run side by side: one cultured in Test medium containing 5 g/L mannose and 5 mM calcium and one cultured in Control medium containing 3 g/L mannose and 1 mM calcium (FIG. 8A). Indeed, an >30% titer benefit was sustained over the perfusion campaign of over 130 days in the Test bioreactor versus the Control (FIG. 8B).

Product quality was tested side by side at three time points for the Test (FIG. 8A, points 3, 5, 7) and the Control (FIG. 8A, points 2, 4, 6) cultures by collecting harvest material from Example 5 and concentrating it by ultra-filtration. An early, time point one (FIG. 8A, point 1) was collected from the Test bioreactor before shifting from control to test medium. The ultra-filtered culture harvest was purified and FVIII quality was assessed. FVIII generated using the Test medium has met all quality metrics including purity and integrity, potency, specific activity, various host-cell impurities and glycosylation patterns; further, FVIII material generated from cultures in Test medium was comparable to that generated from cultures grown in control medium, indicating that the >30% sustained increase in titer did not impact product quality. Cell culture growth attributes were also comparable in the two bioreactors, Test and Control. Process control set points (pH, dissolved oxygen, pCO₂ and temperature), cellular attributes (bioreactor cell density, bioreactor viability), metabolites (residual and consumption rates for glucose and lactate) and specific productivity, were all comparable between the test and control bioreactors.

### Example 6: Material and Methods for Examples 1-5

Roller Tube Experiments

Small scale media testing experiments were carried out in 50 mL culture tubes with vented screw caps (Cultiflask 50, Sartorius, Bohemia NY) as previously described (Shimoni et al., BioPharm International, 23(8): 28-37 (2010)). Tubes were filled with 14 mL of test media with an initial cell density of 3×10⁵ cells/mL. Tubes were mixed in rolling motion at 30 rpm on a rolling tube platform which was placed in a humidified, temperature- and CO₂-controlled incubator. The tubes were incubated for four days, and samples of 1.3 mL were taken for metabolite analysis on days 2, 3 and 4. Additional samples for potency tests with the coagulation or chromogenic assay were taken on days 3 and 4.

1 L Perfusion Bioreactor Cell Culture

For scale up, BHK-21 cells expressing rhFVIII were inoculated in shake flasks using production media (a DMEM/F12 based media). Flasks were incubated at 35.5° C. and 30 rpm and successively split until the desired amount of cells was present.

Cells from scale up were inoculated at 9×10⁶ vc/mL into a 1.5 L DASGIP (Eppendorf, Germany) vessel at a working volume of 1 L on a DASGIP control station. The working volume was kept constant by a level sensor which controlled the media pump.

Perfusion was established using a cell retention device (settler) at a target cell specific perfusion rate (“CSPR”) of 0.45 nL/cell/day at steady state by adjustment of the harvest pump dependent on the measured cell density. Temperature was controlled at 35.5° C. using the station thermostat and the settler temperature was controlled at 20-23° C. Aeration was provided by immersed silicone tubing. Cells were discarded from the bioreactor in response to decreasing dissolved oxygen so as to maintain a target cell density of 25×10⁶ vc/mL. Supplementary aeration was provided by head space aeration of 5 L/hour. Culture pH was controlled at a target of 6.85 by addition of sodium carbonate solution as needed.

15 L Perfusion Bioreactor Cell Culture

Cell culture was conducted in 15 L bioreactors (Applikon Inc., Foster City, Calif.) at a working volume of 12 L. Bioreactors were inoculated at a seeding density of ≧1×10⁶ cells/mL. Standard setpoints for controllable process parameters were maintained throughout the runs; pH=6.8, Temperature=35.5° C., dissolved oxygen DO=50% air saturation. Mixed gas for dissolved oxygen and pH control were supplied to the culture by a silicone membrane and headspace was controlled via a manual rotameter to maintain positive pressure and to aid in stripping. Bioreactors were connected to a cell retention device (settler) to remove cells from the harvest stream and to return the settled mass of cells back to the bioreactor.

CSPR was adjusted to the steady state target of 0.45 nL/cell/day and maintained for the duration of the run. The steady-state cell concentration was targeted at 20×10⁶ vc/mL by automatically discarding cells from the system based on an oxygen flow control algorithm.

Sampling and Sample Processing

Samples from bioreactors and harvest streams were taken daily. Cell concentrations, viabilities and sizes were measured with a Cedex cell counter (Roche Innovatis, Germany). Residual glucose and lactate concentrations were measured with the YSI 2700 biochemical analyzer (YSI Life Sciences, USA). Bioreactor gas and pH were measured with the RapidLab 248 blood gas analyzer (Siemens, Germany). Bioreactor and harvest samples were analyzed for rFVIII quantification by either the one-stage coagulation or chromogenic assay (described below).

FVIII Potency Assays (One-Stage Coagulation and Chromogenic)

The clotting FVIII:C test method is a one-stage assay based upon the activated partial thromboplastin time (aPTT). Factor VIII acts as a cofactor in the presence of Factor IXa, calcium, and phospholipid in the enzymatic conversion of Factor X to Xa. In this assay, the diluted test samples are incubated at 37° C. with a mixture of FVIII deficient plasma substrate and aPTT reagent. Calcium chloride is added to the incubated mixture and clotting is initiated. An inverse relationship exists between the time (seconds) it takes for a clot to form and logarithm of the concentration of FVIII:C. Activity levels for unknown samples are interpolated by comparing the clotting times of various dilutions of test material with a curve constructed from a series of dilutions of standard material of known activity and are reported in International Units per mL (IU/mL).

The chromogenic potency assay method includes two consecutive steps where the intensity of color is proportional to the Factor VIII activity in the sample. In the first step, Factor X is activated to Factor Xa by Factor IXa with its cofactor, Factor VIIIa, in the presence of optimal amounts of calcium ions and phospholipids. Excess amounts of Factor X are present such that the rate of activation of Factor X is solely dependent on the amount of Factor VIII. In the second step, Factor Xa hydrolyzes the chromogenic substrate to yield a chromophore and the color intensity is read photometrically at 405 nm. Potency of an unknown is calculated and the validity of the assay is checked using the linear regression statistical method. Activity is reported in International Units per mL (IU/mL). Further details about the chromogenic and coagulation assays of factor VIII are found in the literature (for reference see: Barrowcliffe T. W. et al., seminars in Thrombosis and Hemostasis, 28 (3), 2002; Lippi G. et al., Blood Coagulation and Fibrinolysis 2009, 20 (1), 2009).

The results of the experiments reported here are given in relative units.

### Ultra-Filtered Culture Harvest

Harvest fluid of the 15 L fermentations was filtered to remove cells and debris and was then concentrated 40 fold by cross flow filtration using a 100 kiloDalton (kDa) cut off membrane.

### Purification to UFDF

rFVIII was purified from the ultra-filtered material by a series of chromatography steps comprising immunoaffinity chromatography by binding of rFVIII to immobilized monoclonal antibodies and ion exchange chromatography as described in Boedeker, Seminars in Thrombosis and Hemostasis, 27(4): 385-394 (2001).

### QC Assays

In order to analyze the quality of the produced rFVIII protein, a series of specific methods were applied. Quality of the FVIII product was assessed for any potential changes in integrity, glycosylation pattern and for host cell impurities.

Factor VIII integrity was analyzed by HPLC. The product was also analyzed for integrity and impurities by silver staining following SDS-PAGE and by Western blots using anti-FVIII antibodies.

For the determination of contaminants and impurities the product was analyzed for host cell proteins using specific immuno assays and also for nucleic acid impurities derived from the BHK cell culture.

The glycosylation pattern of the isolated protein was analyzed by determination of the different sugar components and the degree of sialylation. The data were compared to an in-house control rFVIII protein.

### Concluding Remarks Regarding Examples 1-6

These results demonstrate that it is possible to achieve a titer gain of >30% by introducing and/or increasing mannose and/or calcium concentrations in the culture medium; and the titer gain is sustained for >130 days in continuous perfusion culture. Importantly, neither product quality attributes (including impurities) nor culture performance are impacted by the change to the culture medium. The observed impact on titer increase is not reproduced with glucose, for example, when merely increasing the concentration of glucose in DMEM/F12 without including mannose.

Increasing the mannose concentration from 3 g/L to 5 g/L can increase rhFVIII productivity by over 25% using a 15 L perfusion bioreactor. Independently, a calcium increase from ˜1 mM to 5 mM resulted in almost the same gain in productivity as well. And when mannose and calcium were both increased, the productivity gains further increased to nearly 40%; a combination of 5 g/L mannose and 5 mM calcium yielded >30% increase in Factor VIII specific productivity over standard production medium containing 3 g/L mannose and 1 mM calcium. Cell culture performance and product quality attributes were not impacted by this change to the medium formulation. The impact on productivity is apparent within about a day after media switch and is reversible. Greater than 30% productivity gains were sustained over 3 months from cell bank thaw during continuous perfusion bioreactor cell culture.

The effect of mannose and calcium on titer increase is higher when both are employed, rather than each alone. Calcium is known to stabilize the Factor VIII molecule. The concentration of calcium in the standard DMEM/F12 (1:1) culture medium formulation is only ˜1 mM. Higher levels of calcium in the culture medium formulation can therefore help stabilize the Factor VIII molecule earlier in the process—as early as when Factor VIII is being secreted out of the cells, rather than after the harvest has been collected.

TABLE 1
DMEM:F-12 (1:1) Medium Formulation as described in the ATCC
Catalog
ATCC Catalog No. 30-2006
Inorganic Salts (g/liter)

| | |
|---|---|
| CaCl₂(anhydrous) | 0.11665 |
| CuSO₄(anhydrous) | 0.0000008 |
| Fe(NO₃)₃•9H₂O | 0.00005 |
| FeSO_{4•}7H₂O | 0.000417 |
| MgSO₄(anhydrous) | 0.08495 |
| KCl | 0.3118 |
| NaHC0₃ | 1.20000 |
| NaC1 | 7.00000 |
| Na₂HPO₄(anhydrous) | 0.07100 |
| NaH₂PO₄•H₂O | 0.06250 |
| ZnSO₄•7H₂O | 0.000432 |

Amino Acids (g/liter)

| | |
|---|---|
| L-Alanine | 0.00445 |
| L-Arginine•HCl | 0.14750 |
| L-Asparagine•H₂O | 0.00750 |
| L-Aspartic Acid | 0.00665 |
| L-Cystine•HCl•H₂O | 0.01756 |
| L-Cystine•2HCl | 0.03129 |
| L-Glutamic Acid | 0.00735 |
| L-Glutamine | 0.36510 |
| Glycine | 0.01875 |
| L-Histidine•HCl•H₂O | 0.03148 |
| L-Isoleucine | 0.05437 |
| L-Leucine | 0.05895 |
| L-Lysine-HCl | 0.09135 |
| L-Methionine | 0.01724 |
| L-Phenylalanine | 0.03548 |
| L-Proline | 0.01725 |
| L-Serine | 0.02625 |
| L-Threonine | 0.05355 |
| L-Tryptophan | 0.00902 |
| L-Tryosine•2Na•2H₂O | 0.05582 |
| L-Valine | 0.05285 |

Vitamins (g/liter)

| | |
|---|---|
| D-Biotin | 0.0000036565 |
| Choline Chloride | 0.00898 |
| Folic Acid | 0.00265 |
| myo-inositol | 0.01261 |
| Niacinamide | 0.00202 |
| D-Pantothenic Acid | 0.00224 |
| (hemicalcium) | |
| Pyridoxine•HCl | 0.00203 |
| Riboflavin | 0.00022 |
| Thiamine•HCl | 0.00217 |
| Vitamin B-12 | 0.00068 |

Other (g/liter)

| | |
|---|---|
| D-Glucose | 3.15100 |
| HEPES | 3.57480 |
| Hypoxanthine | 0.00239 |
| Linoleic Acid | 0.000044 |
| Phenol Red, | 0.00810 |
| Sodium Salt | |
| Putrescine•2HCl | 0.00008 |
| Pyruvic Acid•Na | 0.05500 |
| DL-Thioctic Acid | 0.000105 |
| Thymidine | 0.000365 |

The section headings used herein are for organizational purposes only and are not to be construed as limiting the subject matter described in any way.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

The specification and examples are, accordingly, to be regarded in an illustrative rather than a restrictive sense. Furthermore, all articles, books, patent applications and patents referred to herein are incorporated herein by reference in their entireties for all purposes.

The invention claimed is:

1. A mammalian cell culture medium formulation comprising at least one of mannose at about 3.5 g/L or more and calcium in a range from about 1.5 mM to about 9.5 mM.

2. The formulation of claim 1, wherein the medium comprises mannose at about 3.5 g/L or more and calcium at less than about 1.5 mM or more than about 9.5 mM.

3. The formulation of claim 1, wherein the medium comprises mannose at less than about 3.5 g/L and calcium in a range from about 1.5 mM to about 9.5 mM.

4. The formulation of claim 1, wherein the medium comprises mannose in a range from about 4 g/L to about 5 g/L.

5. The formulation of claim 2, wherein the medium comprises mannose in a range from about 4 g/L to about 5 g/L.

6. The formulation of claim 1, wherein the medium comprises calcium in a range from about 2 mM to about 5 mM.

7. The formulation of claim 3, wherein the medium comprises calcium in a range from about 2 mM to about 5 mM.

8. The formulation of claim 1, wherein the formulation comprises DMEM/F12 in 1:1 ratio, and includes at least one of mannose in a range from about 4 g/L to about 5 g/L and calcium in a range from about 2 mM to about 5 mM.

9. A method of producing a recombinant protein in cell culture, comprising:
providing recombinant protein expressing cells; and
culturing the cells in a cell culture medium including at least one of mannose at about 3.5 g/L or more and calcium in a range from about 1.5 mM to about 9.5 mM.

10. The method of claim 9, wherein the medium comprises mannose at about 3.5 g/L or more and calcium at less than about 1.5 mM or more than about 9.5 mM.

11. The method of claim 9, wherein the medium comprises mannose at less than about 3.5 g/L and calcium in a range from about 1.5 mM to about 9.5 mM.

12. The method of claim 9, wherein the medium comprises mannose in a range from about 4 g/L to about 5 g/L.

13. The method of claim 10, wherein the medium comprises mannose in a range from about 4 g/L to about 5 g/L.

14. The method of claim 9, wherein the medium comprises calcium in a range from about 2 mM to about 5 mM.

15. The method of claim 11, wherein the medium comprises calcium in a range from about 2 mM to about 5 mM.

16. The method of claim 9, wherein the cells are mammalian cells.

17. The method of claim 16, wherein the mammalian cells are selected from BHK cells, CHO cells, HKB cells, HEK cells, and NS0 cells.

18. The method of claim 17, wherein the mammalian cells are BHK cells.

19. The method of claim 9, wherein the cells express a blood-coagulation pathway recombinant protein.

20. The method of claim 19, wherein the cells express recombinant human factor VIII (rhFVIII), or a variant thereof.

21. The method of claim 20, wherein the variant factor VIII is a genetic variant.

22. The method of claim 21, wherein the genetic variant is a B-domain deleted FVIII.

23. The method of claim 20, wherein the variant factor VIII is a pegylated FVIII.

24. The method of claim 9, wherein the cells are BHK cells expressing recombinant factor VIII, and wherein the medium comprises DMEM/F12 in 1:1 ratio, and includes at least one of mannose in a range from about 4 g/L to about 5 g/L and calcium in a range from about 2 mM to about 5 mM.
